(19) United States
(12) Patent Application Publication
(10) Pub. No.: US 2017/0232190 A1
(43) Pub. Date: Aug. 17, 2017

(54) INJECTION DEVICE WITH FLASHING INDICATOR

(71) Applicant: CAREBAY EUROPE LTD, Sliema (MT)

(72) Inventor: Farhad BYBORDI, Boynton Beach, FL (US)

(21) Appl. No.: 15/514,722

(22) PCT Filed: Sep. 28, 2015
(86) PCT No.: PCT/EP2015/072268

§ 371 Date: Mar. 27, 2017

(30) Foreign Application Priority Data
Oct. 8, 2014 (SE) 1451200-8

(51) Int. Cl. A61M 5/158; A61M 25/00; A61M 39/24; A61M 5/168; A61M 25/06

(52) U.S. Cl. CPC A61M 5/158; A61M 5/16881; A61M 25/0637; A61M 25/0693; A61M 39/24; A61M 25/0097; A61M 2005/1588; A61M 2039/244

(57) ABSTRACT

A injection device includes: a hub; two wings extending from the hub and functioning as flashing indicators; a tube connected to a first end of the hub; a needle being in fluid communication with the second end of the hub; and a check valve is provided within the tube, wherein a fluid can flow from the tube to the hub through the check valve, and the fluid is prohibited from flowing from the hub to the tube by the check valve. The flashing indicators can be observed by a user to make sure that the needle has been properly inserted into a vein.

### CROSS REFERENCE TO RELATED APPLICATIONS

The present application is a U.S. National Phase Application pursuant to 35 U.S.C. §371 of International Application No. PCT/EP2015/072268 filed Sep. 28, 2015, which claims priority to Swedish Patent Application No. 1451200-8 filed Oct. 8, 2014. The entire disclosure contents of these applications are herewith incorporated by reference into the present application.

### TECHNICAL FIELD

This invention generally relates to an injection device, and more particularly to an injection device with a blood-flashing indicator.

### BACKGROUND

The current practice for intravenous injection requires that one must observe a flash of blood or traces of blood inside the tubing which is attached to a cannula inserted into human or animal vein before starting the injection.

FIG. 1 is an infusion device 1 with a blood-flashing indicator disclosed in U.S. Pat. No. 4,037,600. The infusion device 1 includes a holding body 11, a sheath 12 connected to the proximal end of the holding body 11, a cannula 13 received within the sheath 12, a block 14 connected to the cannula 13 at the distal end thereof, a cavity 15, which functions as the blood-flashing indicator, formed inside the block 14, and a catheter 16 which may be pushed into the sheath 12 for infusing medicine liquid into the vein after the cannula 13 is withdrawn from the sheath 12.

However, in order to accommodate the cannula 13 or catheter 16, the sheath 12, which is used to puncture the skin and vein of a patient, of the infusion device 1 has to be formed with a large diameter. The large diameter makes the patient painful when the sheath 12 punctures the skin and vein.

Furthermore, the operation of the infusion device 1 includes: the steps of withdrawing the cannula 13 out of the sheath 12 and pushing the catheter 16 into the sheath 12. During such two steps, the sheath 12 which has pierced the skin and vein will be moved relative to the skin and vein by a user. The patent will badly suffer from such movement of the sheath 12.

Moreover, the prolonged operation including several steps will detrimentally lengthen patient's duration of pain.

Besides, the complicated infusion device 1 comprised of many components will significantly increase manufacturing cost.

### SUMMARY

Accordingly, this disclosure relates to an injection device with a flashing indicator that is substantially intended to obviate one or more of the problems due to the limitations and disadvantages encountered in the prior art.

One object of this disclosure is to provide an injection device with a flashing indicator in which no sheath or catheter is equipped and thus the diameter of the needle is small.

Another object of this disclosure is to provide an injection device with a flashing indicator whose operation is easy.

Yet another object of this disclosure is to provide an injection device with a flashing indicator which can reduce patient's pain during operation of puncture.

A further object of this disclosure is to provide an injection device with a flashing indicator whose structure is simple.

Another object of this disclosure is to provide an injection device with a flashing indicator, whose cost is low.

These objects are achieved by an injection device with a flashing indicator as defined by claim 1. The dependent claims define preferred or advantageous embodiments of the injection device with a flashing indicator.

Additional features and advantages of the disclosure will be set forth in the description which follows, and in portion will be apparent from the description, or may be learned by practice of the disclosure. The objectives and advantages of the disclosure will be realized and attained by the structure as particularly set forth in the written description and claims as well as illustrated in the appended drawings.

To achieve these and other advantages and according to the purpose of this disclosure, as embodied and broadly described, an injection device comprises: a hollow hub having a periphery, a first end and a second end; two wings extending away from the periphery of the hub; a tube connected to the first end of the hub; a needle being in fluid communication with the second end of the hub; and a check valve is provided within the tube. Fluid can flow from the tube to the hub through the check valve but the fluid is prohibited from flowing from the hub to the tube by the check valve. The injection device includes just one needle but no sheath and catheter so as to reduce steps of injection and simplify the structure of the injection device.

In a preferred aspect, the injection device further comprises a flashing indicator which is in fluid communication with the hub.

Moreover, at least one of the wings is made of transparent material and formed with a chamber therein which functions as the flashing indicator.

Furthermore, the injection device further comprises a first pouch which is made of transparent material, functions as the flashing indicator and connected to the hub.

The flashing indicator provides, for user's observation, indication of appropriate insertion of the needle and can avoid repeating puncture.

It is another preferred feature that the check valve includes at least one flap slantly extending from an inner surface of the tube downward the hub whereby the at least one flap closes the tube when the fluid flows from the hub to the tube.

It is preferred that the check valve includes one flap which contacts a circle of the inner surface of the tube when the flap closes the tube.

It is preferred that the check valve includes two flaps which contact each other when the flaps close the tube.

The check valve can restrict blood trace within the flashing indicator.

Additionally, the chamber in the at least one of the wings is squeezed at the same time as the at least one of the wings is deformed.

It is preferred that the wings are made of resilient material to allow the wings being deformed to the extent that the wings contact each other.

The chambers hidden in the wings can simplify the squeezing operation.

It is preferred that the injection device further comprises a barrel interposed between the hub and the needle.

It is preferred that the injection device further comprises a second pouch which is in fluid communication with the first pouch and is made of transparent material.

It is preferred that the injection device further comprises a branch which is respectively connected to the hub and the first pouch and is in fluid communication with the hub and the first pouch.

It is preferred that the second porch is larger than the first pouch.

It is preferred that at least one of the first porch and the second porch is squeezed before an injection is performed.

It is to be understood that both the foregoing general description and the following detailed description are exemplary and explanatory and are intended to provide a further non-limiting explanation of the disclosure as claimed.

### BRIEF DESCRIPTION OF THE DRAWINGS

The accompanying drawings, which are included to provide a further understanding of the disclosure and constitute a portion of the specification, illustrate embodiments of the disclosure and together with the description serve to explain the principle of the disclosure. In the drawings:

FIG. 1 is a sectional view illustrating a conventional infusion device;

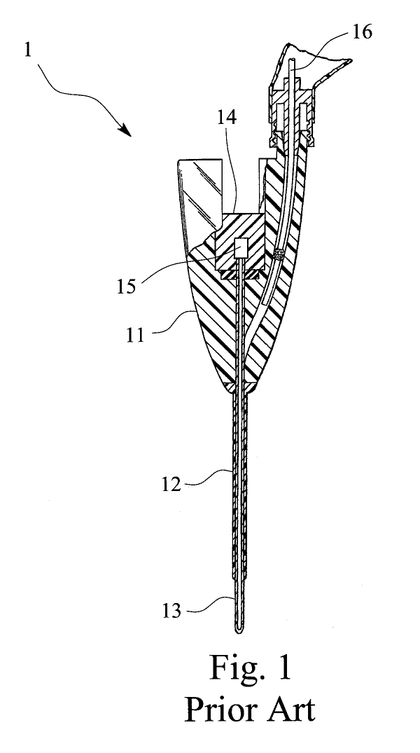

FIG. 2 is a top plan view illustrating the first embodiment of an injection device according to this disclosure;

FIG. 3 a sectional view of FIG. 2 showing that a tube is closed by a check valve;

FIG. 4 a sectional view of FIG. 2 showing that the check valve in the tube is open;

FIG. 5 a plan view illustrating the operation of the injection device according to this disclosure;

FIG. 6 is a top plan view illustrating the second embodiment of an injection device according to this disclosure;

FIG. 7 a sectional view of FIG. 6 showing that a tube is closed by a check valve;

FIG. 8 a sectional view of FIG. 6 showing that the check valve in the tube is open;

### DETAILED DESCRIPTION

### Structure of the First Embodiment

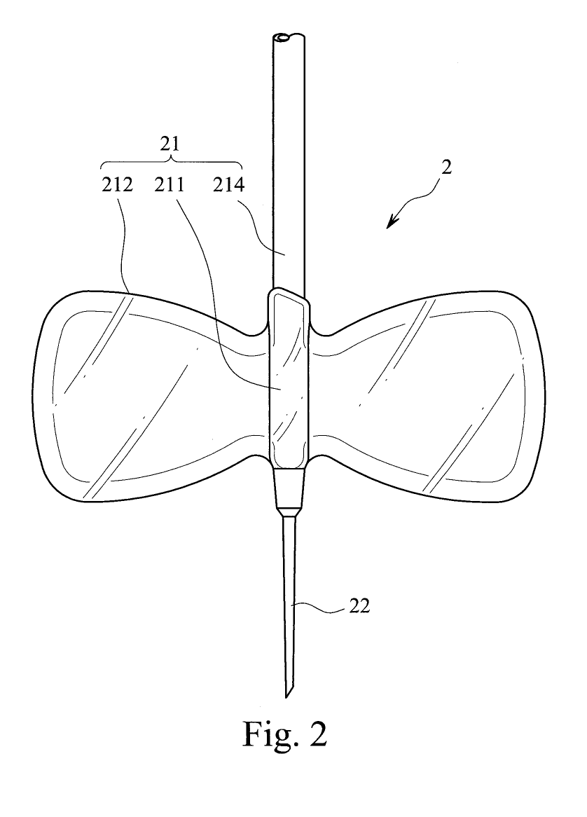

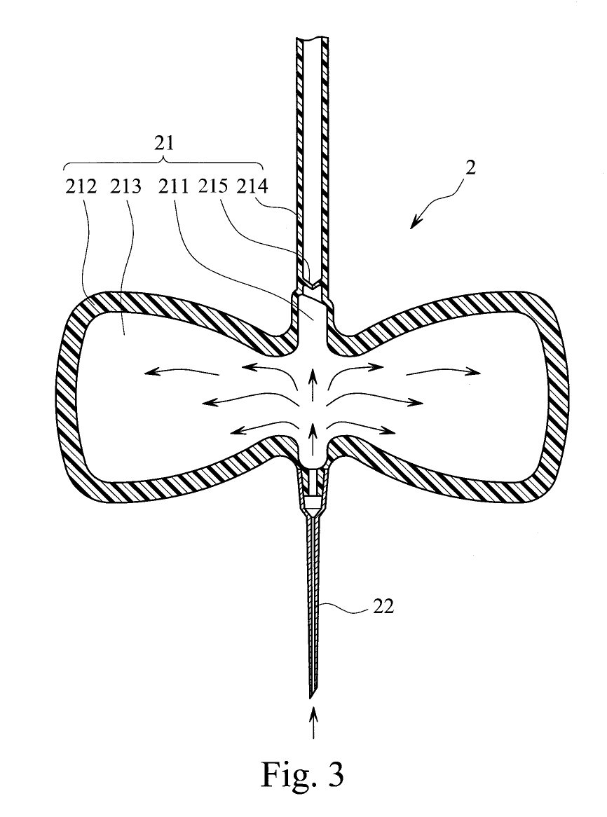

As shown in FIGS. 2 and 3, an injection device 2 comprises a main body 21 and a needle 22 connected to the main body 21.

The main body 21 includes a hollow and cylindrical hub 211, two hollow wings 212 respectively and oppositely extending from periphery of the hub 211, two chambers 213 respectively formed inside the wings 212, a hollow tube 214 extending away from the first end of the hub 211, and a one-way check valve 215 within the tube 214.

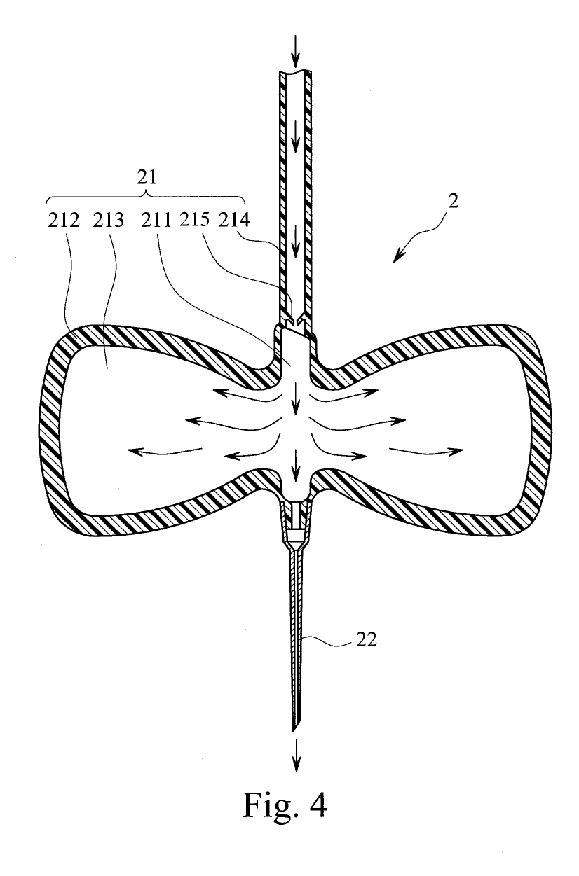

In the first embodiment, the check valve 215 is constituted by two flaps which are made of resilient material such as polyurethane and slantly extend from inner surface, near the joint of the tube 214 and the hub 211, of the tube 214 toward the hub 211. The free ends of two flaps contact each other in common state. Besides, when fluid (such as blood) flows from the hub 211 to the tube 214, the free ends of the two flaps abut against each other so as to close the tube 214 and prevent the fluid from flowing into the tube 214. On the contrary, when fluid (such as medicine, FIG. 4) flows from the tube 214 to the hub 214, the free ends of the flaps are separated by the fluid so as to open the tube 214 and allow the fluid flows into the hub 211.

The wings 212 are formed with transparent and resilient material, which encloses two chambers 213 respectively within the wings 212. The chambers 213 are respectively in fluid communication with the hub 211 and full of air in common state.

The needle 22 is firmly assembled to the second end of the hub 211. As a result, the injection device 2 is generally in a shape of a butterfly.

### Operation of the First Embodiment

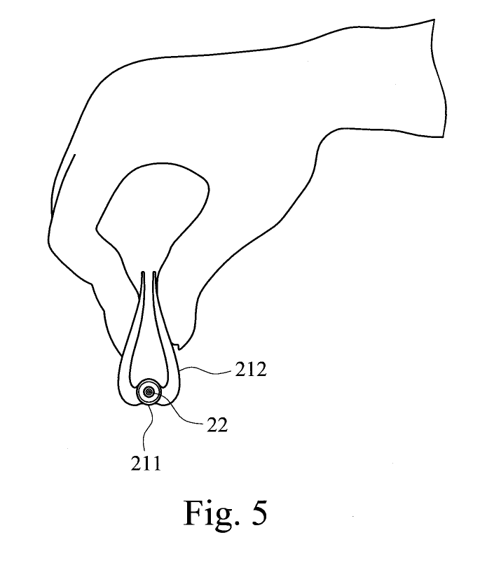

Prior to a puncture, a user may hold the wings 212 with a hand, especially for example with the thumb and index finger, of the injection device 2 to deform the wings 212 around the hub 211 and even to the extent that the wings 212 almost contact each other (FIG. 5). In this state, the profile of the injection device 2 is similar to that of a butterfly swinging its wings to the upmost position. Besides, most air within the chambers 213 is expelled out of the chambers 213 through the hub 211 and needle 22 because the wings 212 are squeezed by the two fingers simultaneously and thus volume of the chambers 213 decreases. It is noted that the air is prevented from flowing into the tube 214 by the check valve 215 in the tube 214.

Then, the user may start the puncture. When the needle 22 is inserted into the skin of the patient, the two fingers may be released a little bit to test whether the needle 22 has been properly inserted into the vein. Because the fingers are slightly released, the resilient material of the wings 212 will recover a little bit and the volume of the chambers 213 increases accordingly. In this state, the pressure of the chambers 213 is lower than the atmosphere and the pressure in the vein. Therefore, if the needle 22 is properly inserted into the vein, blood will flow from the vein to the chambers 213 through the needle 22 and hub 211 for user's observation (FIG. 3). Similarly, the blood is also prevented from flowing into the tube 214 by the check valve 215 in the tube 214.

When the user sees the blood flowing into the chambers 213, which function as flashing indicators, and make sure that the needle 22 has been properly inserted into the vein, injection of medicine from a container (not shown, FIG. 4) connected to the tube 214 may be performed. Alternatively, when the user sees the blood flowing into the chambers 213, the fingers may be entirely released from the wings 212. The resilient material of the wings 212 may completely recover to the extent that the chambers 213 have the largest volume and the chambers will be full of blood (FIG. 3). Subsequently, the wings 212 may be fixed to the skin, for example, by an adhesive tape, if necessary. Then, medicine may flow from the container (not shown, FIG. 4) into the vein through the tube 214, check valve 215, hub 211 and needle 22.

The patient may perform the injection operation with the injection device 2 by himself. In this case, the patient is the user.

### Structure of the Second Embodiment

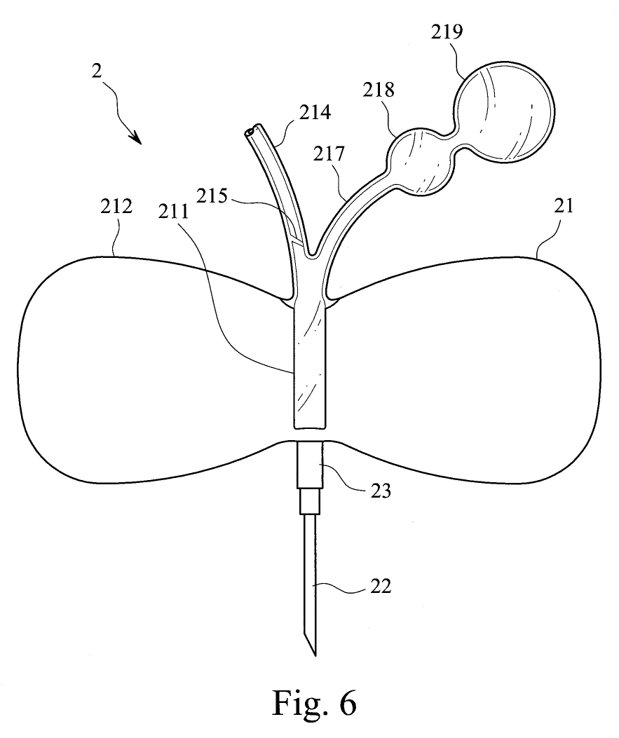

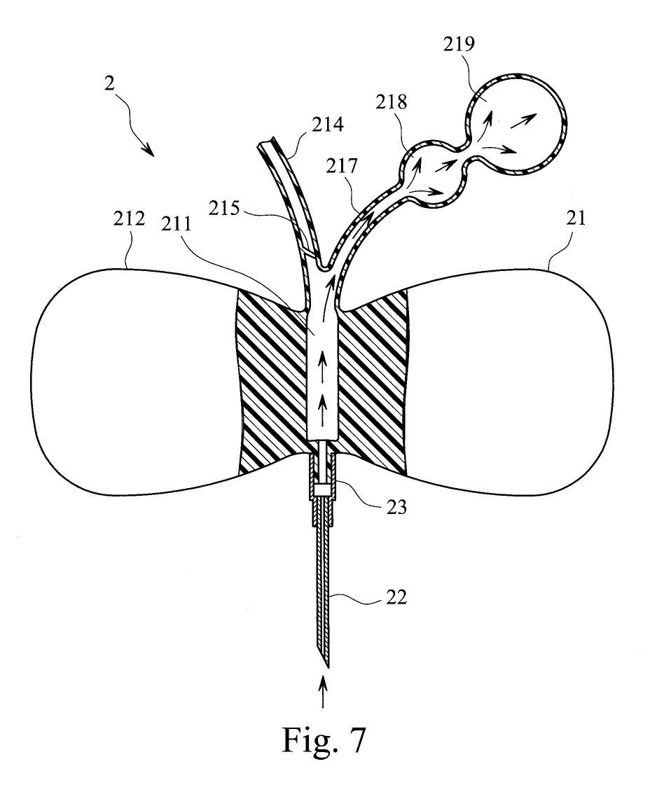

As shown in FIGS. 6 and 7, an injection device 2 comprises a main body 21, a hollow barrel 23 and a needle 22 connected to the main body 21.

The main body 21 includes a hollow and cylindrical hub 211, two solid wings 212 respectively and oppositely extending from periphery of the hub 211 and formed with resilient material, a hollow branch 217 extending away from the first end of the hub 211, a small first pouch 218 extending from the branch 217, a large second pouch 219 extending from the first pouch 218, a hollow tube 214 extending away from the joint of the hub 211 and the branch 217, and a one-way check valve 215 within the tube 214 but outside the joint of the hub 211 and the branch 217. The hub 211, tube 214, branch 217, first pouch 218 and second pouch 218 are in fluid communication one another.

In the second embodiment, the branch 217, first pouch 218 and second pouch 219 constitute a flashing indicator. The flashing indicator is made of transparent and resilient material.

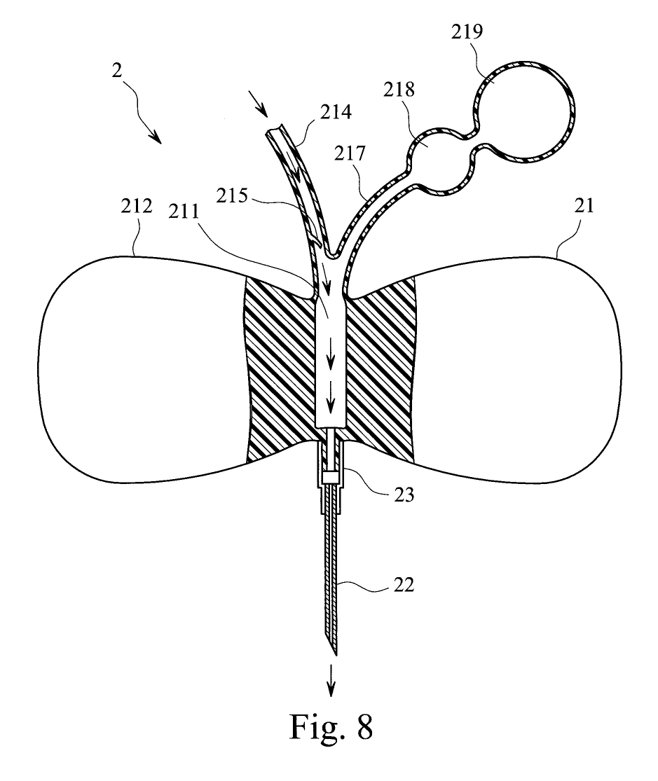

The check valve 215 is constituted by one flap which is made of resilient material such as polyurethane and slantly extends from inner surface, near the joint of the hub 211 and the branch 217, of the tube 214 toward the hub 211. The free end of the flap contacts another portion of the inner surface of the tube 214 in common state. Besides, when fluid (such as blood) flows from the hub 211 to the tube 214, the free end of the flap abuts against the inner surface of the tube 214 so as to prevent the fluid from flowing into the tube 214. On the contrary, when fluid (such as medicine, FIG. 8) flows from the tube 214 to the hub 211, the free end of the flap is separated with the inner surface of the tube 214 by the fluid so as to allow the fluid flows into the hub 211. The check valve 215 cannot control the flow between the hub 211 and the flashing indicator.

The barrel 23 is firmly assembled to the second end of the hub 211. The needle 22 is firmly assembled to another end of the barrel 23. As a result, the injection device 2 is generally in a shape of a butterfly.

Alternatively, the check valve 215 of the second embodiment of the injection device 2 can be substituted by that of the first embodiment of the injection device 2, and vice verse. Besides, the barrel 23 of the second embodiment can be omitted and the needle 22 of the second embodiment may be directly connected the hub 211. On the other hand, the first embodiment may be added with the barrel 22 of the second embodiment. Similarly, the branch 217 of the second embodiment can be omitted and the first pouch 218 of the second embodiment may be directly connected the hub 211.

### Operation of the Second Embodiment

Prior to puncture, a user may hold the wings 212 of the injection device 2 with one hand to deform the wings 212 around the hub 211 and even to the extent that the wings 212 almost contact each other (FIG. 5). In this state, the profile of the injection device 2 is similar to that of a butterfly swinging its wings to the upmost position. The user may simultaneously squeeze the large second pouch 219 or both the first 218 and second pouches 219 of the flashing indicator with another hand, especially for example with the thumb and index finger, to decrease the volume of the pouches 218 and 219 and thus expel air out of the pouches through the hub 211, barrel 23 and needle 22. It is noted that the air is prevented from flowing into the tube 214 by the check valve 215 in the tube 214.

Then, the user may start the puncture. When the needle 22 is inserted into the skin of the patient, the two fingers which are squeezing the pouches 218 and 219 of the flashing indicator may be released a little bit to test whether the needle 22 has been properly inserted into the vein. Because the fingers are slightly released, the resilient material of the porches 218 and 219 will recover a little bit and the volume of the pouches 218 and 219 increases accordingly. In this state, the pressure of the pouches 218 and 219 is lower than the atmosphere and the pressure in the vein. Therefore, if the needle 22 is properly inserted into the vein, blood will flow from the vein to the porches 218 and 219 through the needle 22, barrel 23 and hub 211 for user's observation (FIG. 7). Similarly, the blood is also prevented from flowing into the tube 214 by the check valve 215 in the tube.

When the user sees the blood flowing into the porches 218 and 219, which function as flashing indicators, and make sure that the needle 22 has been properly inserted into the vein, injection of medicine from a container (not shown, FIG. 8) connected to the tube 214 may be performed. Alternatively, when the user sees the blood flowing into the porches 218 and 219 (FIG. 7), the fingers may be entirely released from the porches 218 and 219. The resilient material of the porches 218 and 219 may completely recover to the extent that the porches 218 and 219 have the largest volume and the porches 218 and 219 will be full of blood (FIG. 7). Subsequently, another hand may be released from the wings 212 and the wings 212 may be fixed to the skin, for example, by an adhesive tape, if necessary. Then, medicine may flow from a container (not shown, FIG. 8) into the vein through the tube 214, check valve 215, hub 211, barrel 23 and needle 22.

The patient may perform the injection operation with the injection device 2 by himself. In this case, the patient is the user.

### Effects

The first embodiment of the injection device 2 is arranged with two chambers 213 in the wings 212 functioning as blood-flashing indicators. Such arrangement is more reliable than the traditional indicator with only one chamber.

The second embodiment of the injection device 2 is arranged with two pouches 218 and 219 functioning as blood-flashing indicators. Such arrangement is more reliable that the traditional indicator with only one pouch.

The second pouch 219 of the second embodiment is formed with a larger volume than that of the first pouch 218 which is closer to the hub. Such arrangement creates lower pressure in the second pouch 219 than that in the first pouch 218 to make sure blood can flow into the second pouch 219.

The injection device 2 with a flashing indicator of this disclosure is not equipped with a sheath or catheter and thus the diameter of the needle 22 is small.

Because there are no steps for operating the sheath and catheter, the needle 22 can be directly inserted into the vein for observing the flashing blood and injecting medicine. Hence, the operation of the injection device 2 with a flashing indicator of this disclosure is easy.

Because steps for injection are reduced, duration for injection is shortened and movement of the needle is avoided during injection. Hence, patient's pain can be reduced.

Because there are no sheath and catheter, the structure of the injection device 2 with a flashing indicator of this disclosure is simple and the cost of the injection device 2 is low.

This disclosure has been disclosed in terms of specific embodiments. It will be apparent that many modifications can be made to the disclosed structures without departing from the disclosure. Therefore, it is the intent of the appended claims to cover all such variations and modifications that are within the scope of this disclosure.

1. An injection device comprising:
a hollow hub having a periphery, a first end and a second end;
two wings extending away from the periphery of the hub;
a tube connected to the first end of the hub;
a needle being in fluid communication with the second end of the hub; and
a check valve positioned within the tube, wherein a fluid can flow from the tube to the hub through the check valve, and the fluid is prohibited from flowing from the hub to the tube by the check valve.

2. The injection device according to the claim 1, further comprising a flashing indicator which is in fluid communication with the hub.

3. The injection device according to the claim 2, wherein at least one of the wings is made of transparent material and formed with a chamber therein which functions as the flashing indicator.

4. The injection device according to the claim 2, further comprising a first pouch which is made of transparent material, functions as the flashing indicator and connected to the hub.

5. The injection device according to the claim 3, wherein the check valve includes at least one flap slantly extending from an inner surface of the tube downward the hub whereby the at least one flap closes the tube when the fluid flows from the hub to the tube.

6. The injection device according to the claim 5, wherein the check valve includes one flap which contacts a circle of the inner surface of the tube when the flap closes the tube.

7. The injection device according to the claim 5, wherein the check valve includes two flaps which contact each other when the flaps close the tube.

8. The injection device according to the claim 5, wherein the chamber in the at least one of the wings is squeezed at the same time as the at least one of the wings is deformed.

9. The injection device according to the claim 8, wherein the wings are made of resilient material to allow the wings being deformed to the extent that the wings contact each other.

10. The injection device according to the claim 9, further comprising a barrel interposed between the hub and the needle.

11. The injection device according to the claim 4, wherein the check valve includes at least one flap slantly extending from an inner surface of the tube downward the hub whereby the flap closes the tube when the fluid flows from the hub to the tube.

12. The injection device according to the claim 11, wherein the check valve includes one flap which contacts a circle of the inner surface of the tube when the flap closes the tube.

13. The injection device according to the claim 11, wherein the check valve includes two flaps which contact each other when the flaps close the tube.

14. The injection device according to the claim 11, wherein the wings are made of resilient material to allow the wings being deformed to the extent that the wings contact each other.

15. The injection device according to the claim 14, further comprising a second pouch which is in fluid communication with the first pouch and is made of transparent material.

16. The injection device according to the claim 15, further comprising a branch which is respectively connected to the hub and the first pouch and is in fluid communication with the hub and the first pouch.

17. The injection device according to the claim 16, wherein the second porch is larger than the first pouch.

18. The injection device according to the claim 17, wherein at least one of the first pouch and the second porch is squeezed before an injection is performed.

19. The injection device according to the claim 17, further comprising a barrel interposed between the hub and the needle.

20. An injection device comprising:
a hollow hub having a periphery, a first end and a second end;
two wings extending from the periphery of the hub;
a tube connected to the first end of the hub;
a needle being in fluid communication with the second end of the hub; and
a check valve positioned within the tube,
wherein the wings are made of transparent material formed as two chambers each having a first volume and configured to each have a second volume when the wings are squeezed, where the second volume is less than the first volume.
